(19) United States
(12) Patent Application Publication
(10) Pub. No.: US 2013/0028977 A1
(43) Pub. Date: Jan. 31, 2013

(54) PHARMACEUTICAL POWDER COMPOSITION FOR INHALATION

(71) Applicants: Ulhas Dhuppad, Nashik (IN); Sunil Sudhakar Chaudhari, Nashik (IN)

(72) Inventors: Ulhas Dhuppad, Nashik (IN); Sunil Sudhakar Chaudhari, Nashik (IN)

(73) Assignee: GLENMARK PHARMACEUTICALS LIMITED, MUMBAI (IN)

(21) Appl. No.: 13/638,583

(22) PCT Filed: Mar. 30, 2011
(86) PCT No.: PCT/IB2011/000674

§ 371 Date: Sep. 28, 2012

(30) Foreign Application Priority Data
Mar. 31, 2010 (IN) 1082/MUM/2010

(51) Int. Cl. A61K 9/14; A61K 31/137; A61P 11/14; A61P 29/00; A61P 11/00; A61K 31/56; A61P 11/06

(52) U.S. Cl. 424/490; 514/171; 514/653

(57) ABSTRACT

The present invention relates to a pharmaceutical powder composition for inhalation comprising an active ingredient and a pharmaceutically acceptable carrier, process for preparing such composition, and its use for the treatment of respiratory disorder in a subject.

### PRIORITY

This patent application claims priority to an Indian provisional patent application number 1082/MUM/2010 filed on Mar. 31, 2010, the contents of which are incorporated by reference herein.

### TECHNICAL FIELD

The present patent application relates to a pharmaceutical powder composition for inhalation. Particularly, the present patent application relates to a pharmaceutical powder composition for inhalation comprising an active ingredient and a pharmaceutically acceptable carrier, process for preparing such composition, and its use for the treatment of respiratory disorder in a subject.

### BACKGROUND

Traditionally, inhalation therapy has played a relatively minor role in the administration of conventional pharmaceuticals when compared to more conventional drug administration routes, such as oral and intraveneous. However, oral and intravenous routes have many disadvantages, and alternative administration routes are needed. Inhalation is one such alternative administration route. For example, in pulmonary administration a particulate medicament composition is inhaled by the patient. The various alternatives to attain inhalation type compositions are nebulizers, pressurized metered dose inhalers (pMDIs) and dry powder inhalers (DPIs).

DPIs can be basically divided into two types, i.e. single dose inhalers and multiple dose inhalers. Dry powder formulations, while offering unique advantages over cumbersome liquid dosage forms and propellant-driven formulations, are prone to aggregation and low flowability phenomena which considerably diminish the efficiency of dry powder-based inhalation therapies. PCT patent application publication numbers WO9831352, WO2003074036, WO9602231 disclose various dry powder formulations for inhalation.

The efficacy of a dry powder inhaler (DPI) is related to the extent of the drug's deposition in the lungs, which in turn depends on the drug formulation and the device being used. With a DPI, delivery of the defined drug dose to the lung relies on control of the aerosolisation process initiated by the inhaling patient.

During inhalation, air is inhaled through a dose causing it to fluidise and aerosolise to form a cloud of particles that is drawn into the body. Effective fluidisation releases the entire dose to the user. Very fine particles deposit in the lung while coarser particles are retained in the throat and subsequently ingested.

The fraction of a dose that deposits in the lungs, because of its size, is often referred to as the fine particle fraction (FPF), or fine particle dose (FPD). With the conventional formulation, only 10-20% of the emitted dose is deposited in the alveolus. The major issue in inhalation of dry powder drugs is its low efficiency.

The key to better efficiency is the direct correlation between specific powder characteristics and DPI performance. Formulations for use in DPIs need to fluidise and aerosolise in an appropriate way to ensure effective drug delivery to the lung.

There still exists a need for an inhalable composition that provides an efficient delivery of defined dose of active to the lungs.

### SUMMARY

The present invention relates to a pharmaceutical powder composition for inhalation comprising an active ingredient and a pharmaceutically acceptable carrier.

In an embodiment, the present invention provides a pharmaceutical powder composition for inhalation comprising an active and a pharmaceutically acceptable carrier wherein the active ingredient is at least partially coated onto the carrier, and the composition has a deposition of emitted dose (“DED”) in the range of about 20% to about 75%.

In a preferred embodiment, the composition has a deposition of emitted dose in the range of about 22% to about 70%, or more preferably, in the range of about 25% to about 60%.

In another embodiment, the present invention relates to a pharmaceutical powder composition for inhalation comprising an active ingredient and a pharmaceutically acceptable carrier, wherein the active ingredient is at least partially coated onto the carrier, and the composition has a fine particle dose (“FPD”) in the range of about 20% to about 75%. Preferably, the composition has a fine particle dose in the range of about 22% to about 70%, and more preferably, in the range from about 25% to about 60%.

In the context of present invention, the active ingredient is selected from the group consisting of beta-2 adrenergic agonists, steroids, anti-cholinergics, mucolytics and combinations thereof. Preferably, the active ingredient includes but is not limited to salbutamol, salmeterol, terbutaline, metaproterenol, formoterol, fenoterol, procaterol, bitolterol, pirbuterol, fluticasone, budesonide, mometasone, beclomethasone, ciclesonide, acetylcysteine, ambroxol, bromhexine, carbocisteine, erdosteine, mesna, tiotropium, ipratropium, aclidinium or combinations thereof.

More preferably, the active ingredient is selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof.

In an embodiment, the pharmaceutically acceptable carrier includes lactose, mannitol, sucrose, trehalose cyclodextrin, or mixtures thereof. Preferably, the pharmaceutically acceptable carrier is lactose.

In another embodiment, the present invention relates to a pharmaceutical powder composition for inhalation, wherein the weight ratio of the active ingredient to the carrier ranges from about 1:0.1 to about 1:1000. Preferably, the weight ratio of the active ingredient to the carrier ranges from about 1:2 to about 1 to 500, and more preferably, from about 1:50 to about 1 to 200.

In an embodiment, the present invention relates to a pharmaceutical powder composition for inhalation comprising an active ingredient selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof, and lactose, wherein the active ingredient is at least partially coated onto said lactose, and the composition has a deposition of emitted dose in the range of about 20% to about 75%. Preferably, the composition has a deposition of emitted dose in the range of about 22% to about 70%, and more preferably, in the range of about 25% to about 60%.

In a further embodiment, the present invention relates to a pharmaceutical powder composition for inhalation comprising an active ingredient selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof, and lactose, wherein the active ingredient is at least partially coated onto said lactose, and the composition has a fine particle dose in the range of about 20% to about 75%. Preferably, the composition has a fine particle dose in the range of about 22% to about 70%, and more preferably, in the range of about 25% to about 60%.

In an embodiment, the pharmaceutical powder composition of the present invention may be further be filled into a capsule for inhalation or may be processed into a lightly compressed tablet or powder agglomeration which can be easily crushed to obtain a powder for inhalation. Alternately, the composition can be filled, as discrete dosage units, in a blister or a sachet.

In an embodiment, the present invention relates to a process for preparing a pharmaceutical powder composition for inhalation, said process comprising: (a) coating the dispersion of active onto the pharmaceutically acceptable carrier to obtain a powder composition; (b) sizing the powder composition and blending the powder composition.

In another embodiment, the present invention relates to a process for preparing a pharmaceutical powder composition for inhalation, said process comprising: (a) dispersing the active ingredient in a solvent; (b) coating the dispersion onto the pharmaceutically acceptable carrier to obtain a powder composition; (c) sizing the powder composition; and (d) optionally, blending the powder composition with the pharmaceutically acceptable carrier.

In an embodiment, the present invention relates to a pharmaceutical powder composition for the treatment of respiratory disorder in a subject comprising administering by inhalation to the subject an effective amount of a pharmaceutical powder composition comprising an active and a pharmaceutically acceptable carrier wherein the active is at least partially coated onto the carrier, and the composition has a deposition of emitted dose in the range of about 20% to about 75%.

In another embodiment, the present invention provides a method for treating a respiratory disorder in a subject, the method comprising administering by inhalation to the subject, an effective amount of a pharmaceutical powder composition comprising an active and a pharmaceutically acceptable carrier wherein the active is at least partially coated onto the carrier, and the composition has a deposition of emitted dose in the range of about 20% to about 75%.

In yet another embodiment, the present invention relates to use of an effective amount of pharmaceutical powder composition for the treatment of respiratory disorder, wherein the composition comprises an active and a pharmaceutically acceptable carrier wherein the active is at least partially coated onto the carrier, and the composition has a deposition of emitted dose in the range of about 20% to about 75%.

The respiratory disorder includes but is not limited to airway inflammation, asthma, emphysema, bronchitis, chronic obstructive pulmonary disease, sinusitis, rhinitis, cough, respiratory depression, reactive airways dysfunction syndrome (RADS), acute respiratory distress syndrome (ARDS), irritant induced asthma, occupational asthma, sensory hyper-reactivity, multiple chemical sensitivity, and aid in smoking cessation therapy.

### BRIEF DESCRIPTION OF DRAWINGS

FIG. 1 represents a typical sectional view showing an inhaler device as used in the context of the present invention for determination of deposition of emitted dose and fine particle dose.

### DETAILED DESCRIPTION

The terms used herein are defined as follows. If a definition set forth in the present application and a definition set forth earlier in a provisional application from which the priority is claimed are in conflict, the definition in the present application shall control the meaning of the terms.

The term “effective amount” or “therapeutically effective amount” denotes an amount of an active ingredient that, when administered to a subject for treating a state, disorder or condition, produces an intended therapeutic benefit in a subject.

The term “active” (used interchangeably with “active ingredient” or “active substance” or “drug”) as used herein includes a drug that is effective in the treatment of respiratory disorders and include a prodrug, ester, salt or other derivative of the drug.

By “salts” or “pharmaceutically acceptable salts”, it is meant those salts and esters which are, within the scope of sound medical judgment, suitable for use in contact with the tissues of humans and lower animals without undue toxicity, irritation, and allergic response, commensurate with a reasonable benefit to risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, xinafoate and lauryl sulphate salts. Representative alkali or alkaline earth metal salts include the sodium, calcium, potassium and magnesium salts.

The term “treating” or “treatment” as used herein also covers the prophylaxis, mitigation, prevention, amelioration, or suppression of a disorder modulated by a mucolytic, or a beta-2 adrenergic receptor agonist or steroid or anticholinergic agent, or by their combinations in a mammal.

By the term “respiratory disorder”, it is meant to any condition or disease related to respiration or the respiratory system and includes but not limited to airway inflammation, asthma, emphysema, bronchitis, chronic obstructive pulmonary disease, sinusitis, rhinitis, cough, respiratory depression, reactive airways dysfunction syndrome (RADS), acute respiratory distress syndrome (ARDS), irritant induced asthma, occupational asthma, sensory hyper-reactivity, multiple chemical sensitivity, and aid in smoking cessation therapy.

The term, “pharmaceutical powder composition for inhalation” as used herein refers to a particulate formulation that is to be administered to a subject in need thereof by inhalation or by nasal route. The term also includes compositions that are easily reduced to particulate form before inhalation and include but are not limited to lightly compressed tablets, powder agglomerates, capsules filled with a particulate formulation, and the like.

The term “subject” includes mammals like human and other animals, such as domestic animals (e.g., household pets including cats and dogs) and non-domestic animals (such as wildlife). Preferably, the subject is a human.

By “pharmaceutically acceptable excipients”, it is meant any of the components of a pharmaceutical composition other than the actives and which are approved by regulatory authorities or are generally regarded as safe for human or animal use.

By the term, “at least partially coated” as used in the context of the present invention refers to a composition wherein the drug(s), in the form of a dispersion or a solution, have been coated or adsorbed onto the surface of the carrier such that the drug at least partially covers the surface of the carrier, but not merely admixed with the carrier. The inventors of the present invention have surprisingly discovered that a pharmaceutical powder composition wherein the active ingredient is at least partially coated onto the carrier has better properties (e.g., deposition of emitted dose, and fine particle dose) compared to the compositions wherein the active is simply admixed with the carrier.

As used herein, the term “deposition of emitted dose” (“DED”) refers to the proportion of the total emitted dose (excluding the dose deposited in the actuator and upper impingement chamber) that is believed to be deposited in the lungs as assessed by an in vitro method using the Apparatus A—Glass Impinger of the British Pharmacopoeia 2010 (A 324 Appendix XII C) at a flow rate of 60±5 litres/min.

The term “fine particle dose” (synonymously “FPD”) means the fraction of particles being less that about 5 μm when assessed by a cumulative plot from data derived from the use of Apparatus D—Andersen Cascade Impactor of British Pharmacopoeia 2010 (A 330 Appendix XII C) at a flow rate of 60±5 litres/min.

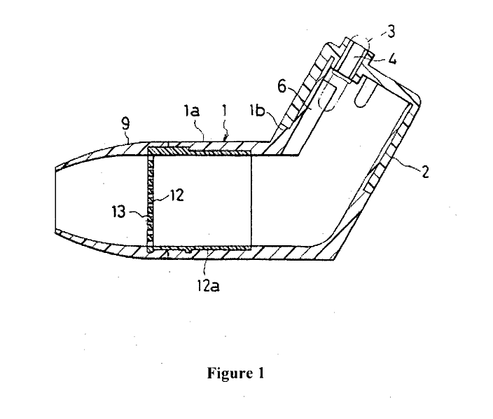

Generally, it is believed that the DED and the FPD parameters are governed by the powder composition in conjunction with a specific inhalation device. In the context of present invention, these parameters were typically determined for various powder compositions using an inhalation device as depicted in FIG. 1.

The term “inhalation device” (used interchangeably with “device” or “inhaler device”) as used in the context of the present invention refers to the inhaler as depicted in FIG. 1. A very similar inhalation device has been prior disclosed in U.S. Pat. No. 4,846,168.

Referring to FIG. 1, the inhaler device has a hollow body (I) including a hollow barrel section (1a) and a straight-lined hollow bent section (1b) formed integrally with this hollow barrel section (1a) and having an inclined axis. A generally capsule holding portion (2) is mounted on one end of the bent section 1b of the hollow body (1) in a manner to be rotatable relative to the hollow body (1). As indicated by dotted lines in FIG. 1, formed at the end face of the capsule holding portion (2) is a capsule insertion hole (4) for receiving and holding a capsule (3) containing the composition of the present invention. A capsule head is inserted into the capsule insertion hole (4) and held therein. A capsule barrel projects into the bent section 1b of the hollow body (1). An abutting ridge (abutting means) (6) extending at least to a position capable of abutting against the portion of capsule (3) that is inserted and projected into the bent section (1b), i.e. the capsule barrel, when the capsule (3) is inserted into the capsule insertion hole (4) and held thereby, is projectingly provided and formed integrally at a portion of the inner surface of the bent section (1b) of the hollow body (1). Under this arrangement, when the capsule holding portion (2) is rotated relative to the hollow body (1), the abutting ridge (6) abuts against the portion of capsule (3) that is inserted and projected into the device, i.e. the capsule barrel, whereby the capsule barrel is separated from the capsule head, so that the powdered preparations in the capsule (3) can fall into the device. On the other hand, detachably, couplingly connected to an end portion of the hollow barrel section (1a) of the hollow body (1) is a hollow inhaling section (9) to be held in the mouth of the patient. The patient at the inhaling section (9) takes in air and inhales the powdered preparations which have fallen from the capsule (3) into the device. Furthermore, a porous plate (12) (capsule discharging preventive means) is connected to an end face of the hollow body (1) on the side of the inhaling section (9). The porous plate (12) is integrally formed on a cylindrical portion (12a) to provide a cup-shaped member. This porous plate (12) has a multitude of pores (13) for ventilating. The pores (13) may have such a size as to allow the powdered preparations as being the material contained in the capsule (3) to pass therethrough freely but not to allow the capsule barrel which has fallen into the hollow body (1) to be discharged to the outside of the device.

Thus, in an embodiment, the present invention relates to a pharmaceutical powder composition for inhalation comprising an active ingredient and a pharmaceutically acceptable carrier.

In another embodiment, the present invention provides a pharmaceutical powder composition for inhalation comprising an active and a pharmaceutically acceptable carrier wherein the active ingredient is at least partially coated onto the carrier, and the composition has a deposition of emitted dose (“DED”) in the range of about 20% to about 75%.

In a preferred embodiment, the composition has a deposition of emitted dose in the range of about 22% to about 70%, or more preferably, in the range of about 25% to about 60%.

In another embodiment, the present invention relates to a pharmaceutical powder composition for inhalation comprising an active ingredient and a pharmaceutically acceptable carrier, wherein the active ingredient is at least partially coated onto the carrier, and the composition has a fine particle dose (“FPD”) in the range of about 20% to about 75%. Preferably, the composition has a fine particle dose in the range of about 22% to about 70% or from about 25% to about 60%.

In the context of present invention, the active ingredient is selected from the group consisting of beta-2 adrenergic agonists, steroids, anti-cholinergics, mucolytics and combinations thereof. Preferably, the active ingredient includes but is not limited to salbutamol, salmeterol, terbutaline, metaproterenol, formoterol, fenoterol, procaterol, bitolterol, pirbuterol, fluticasone, budesonide, mometasone, beclomethasone, ciclesonide, acetylcysteine, ambroxol, bromhexine, carbocisteine, erdosteine, mesna, tiotropium, ipratropium, aclidinium or combinations thereof.

More preferably, the active ingredient is selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof.

In an embodiment, the pharmaceutically acceptable carrier includes lactose, mannitol, sucrose, trehalose cyclodextrin, or mixtures thereof. Preferably, the pharmaceutically acceptable carrier is lactose.

In another embodiment, the present invention relates to a pharmaceutical powder composition for inhalation, wherein the weight ratio of the active ingredient to the carrier ranges from about 1:0.1 to about 1:1000. Preferably, the weight ratio of the active ingredient to the carrier ranges from about 1:2 to about 1 to 500, and more preferably, from about 1:50 to about 1 to 200.

In an embodiment, the present invention relates to a pharmaceutical powder composition for inhalation comprising an active ingredient selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof; and lactose, wherein the active ingredient is at least partially coated onto said lactose, and the composition has a deposition of emitted dose in the range of about 20% to about 75%. Preferably, the composition has a deposition of emitted dose in the range of about 22% to about 70%, and more preferably, in the range of about 25% to about 60%.

In a further embodiment, the present invention relates to a pharmaceutical powder composition for inhalation comprising an active ingredient selected from a group consisting of salbutamol, salmeterol. formoterol, fenoterol, fluticasone, budesonide, mometasone, beclomethasone, ciclesonide, ambroxol, tiotropium, ipratropium, aclidinium or combinations thereof; and lactose, wherein the active ingredient is at least partially coated onto said lactose, and the composition has a fine particle dose in the range of about 20% to about 75%. Preferably, the composition has a fine particle dose in the range of about 22% to about 70%, and more preferably, in the range of about 25% to about 60%.

In an embodiment, the pharmaceutical powder composition of the present invention may be further filled into a capsule for inhalation or may be processed into a lightly compressed tablet or powder agglomeration which can be easily crushed to obtain a powder for inhalation. Alternately, the composition can be filled, as discrete dosage units, in a blister or a sachet.

In an embodiment, the pharmaceutical powder composition of the present invention is filled into capsules for inhalation. The capsule may be a hard gelatin capsule or a cellulose capsule that can be easily broken or opened using an inhalation device.

In another embodiment, the powder composition of the present invention may be in the form of a lightly compressed tablet or powder agglomeration which can be easily crushed to obtain a powder.

The powder composition of the present invention may display a bulk density of from about 0.1 to 1.0 g/ml.

The powder composition of the present invention may contain one or more pharmaceutically acceptable excipients in addition to the carrier. Examples of such excipients include but are not limited to glidants, anti-static agents, solvents, and the like.

The glidants suitable for use in the invention include but are not limited to magnesium stearate, talc, calcium stearate and the like.

Examples of solvents include water; tetrahydrofuran; propylene glycol; ether; petroleum ether; alcohols, e.g., methanol, ethanol, isopropyl alcohol and higher alcohols; alkanes, e.g., pentane, hexane and heptane; ketones, e.g., acetone and methyl ethyl ketone; chlorinated hydrocarbons, e.g., chloroform, carbon tetrachloride, methylene chloride and ethylene dichloride; acetates, e.g., ethyl acetate and the like and mixtures thereof.

In an embodiment, the present invention relates to a pharmaceutical powder composition for the treatment of respiratory disorder in a subject comprising administering by inhalation to the subject a effective amount of a pharmaceutical powder composition comprising an active and a pharmaceutically acceptable carrier wherein the active is at least partially coated onto the carrier, and the composition has a deposition of emitted dose in the range of about 20% to about 75%.

In another embodiment, the present invention provides a method for treating a respiratory disorder in a subject, the method comprising administering by inhalation to the subject, an effective amount of a pharmaceutical powder composition comprising an active and a pharmaceutically acceptable carrier wherein the active is at least partially coated onto the carrier, and the composition has a deposition of emitted dose in the range of about 20% to about 75%.

In yet another embodiment, the present invention relates to use of a effective amount of pharmaceutical powder composition for the treatment of respiratory disorder, wherein the composition comprises an active and a pharmaceutically acceptable carrier wherein the active is at least partially coated onto the carrier, and the composition has a deposition of emitted dose in the range of about 20% to about 75%.

In another embodiment, the present invention relates to a process for preparing a pharmaceutical powder composition comprising coating the dispersion of active onto the pharmaceutically acceptable carrier to obtain a powder composition; and sizing the powder composition and blending it.

In another embodiment, the present invention relates to a process for preparing a pharmaceutical powder composition for inhalation, said process comprising: (a) dispersing the active ingredient in a solvent; (b) coating the dispersion onto the pharmaceutically acceptable carrier to obtain a powder composition; (c) sizing the powder composition; and (d) optionally, blending the powder composition with the pharmaceutically acceptable carrier.

Alternately, the actives may be dissolved into a solvent before loading onto the pharmaceutically acceptable carrier.

The powder composition may be further be filled into a capsule for inhalation or may be processed into a lightly compressed tablet or powder agglomeration which can be easily crushed to obtain a powder for inhalation.

Alternately, the composition can be filled, as discrete dosage units, in a blister or a sachet.

Alternately, the process comprises:
a) sizing the drug particles and carrier to the desired size range;
b) dispersing the drugs together or separately in suitable dispersion medium;
c) layering the drug dispersion onto the carrier particles in a suitable equipment; and
d) drying the layered mass and sizing it.

It will be understood that various modifications may be made to the embodiments disclosed herein. Therefore the above description should not be construed as limiting, but merely as exemplifications of preferred embodiments. Other arrangements and methods may be implemented by those skilled in the art without departing from the scope and spirit of this invention.

All publications, patents, and patent applications cited in this application are herein incorporated by reference to the same extent as if each individual publication, patent, or patent application was specifically and individually indicated to be incorporated herein by reference.

The following examples are provided to enable one skilled in the art to practice the invention and are merely illustrative of the invention. The examples should not be read as limiting the scope of the invention.

### EXAMPLES

### Examples 1-3

### Pharmaceutical Inhalation Compositions Containing Ambroxol Hydrochloride and Salbutamol Sulphate

| | Quantity (% w/w) | | |
|---|---|---|---|
| Ingredients | Example 1 | Example 2 | Example 3 |
| Ambroxol | 8 | 16 | 24 |
| hydrochloride | | | |
| Salbutamol sulfate | 0.48 | 0.48 | 0.48 |
| Lactose monohydrate | 90 | 85 | 75 |
| (Respitose SV010) | | | |
| Ethanol | q.s. | q.s. | q.s. |
| Water | q.s. | q.s. | q.s. |

Manufacturing process:
1. Salbutamol sulphate was dispersed in a sufficient quantity of water.
2. Ambroxol hydrochloride was dispersed in a sufficient quantity of ethanol.
3. Lactose monohydrate was loaded onto a fluidized bed processor and the drug dispersions of Step 1 and Step 2 were coated onto the lactose monohydrate particles by top spray process to obtain granules.
4. The granules of Step 3 were dried and sized through ASTM Sieve # 200 to obtain particles.
5. The particles of Step 4 were filled in hard gelatin capsules so that each capsule contains 0.12 mg of salbutamol sulphate.

The compositions of Examples 1-3 were evaluated for deposition of emitted dose (DED) and fine particle dose (FPD) of salbutamol.

| | Deposition of | Fine particle |
|---|---|---|
| Composition | emitted dose (%) | dose (%) |
| Example 1 | 23.70 | 26.00 |
| Example 2 | 24.80 | 23.00 |
| Example 3 | 22.60 | 27.00 |

Brief analytical procedure:
i. Deposition of Emitted Dose: The DED was calculated using the Apparatus A—Glass Impinger of the British Pharmacopoeia 2010 (A 324 Appendix XII C) The airflow was adjusted to 60±5 litres/min. The capsule containing the composition was broken in the inhalation device (as described in FIG. 1) and was located in the mouthpiece of the Apparatus. The pump was switched on for 5 seconds. After switching off the pump, the device was removed. The discharge sequence was repeated 4 times. The inner surface of the inlet tube and the outer surface of the tube that projects into the lower impingement chamber were washed with the diluent and collected in the lower impingement chamber. The mixture of equal volumes of water and acetonitrile was used as the diluent. The washings were diluted to 100 ml with the diluent and filtered through 0.45 μm filter and subjected to analysis.

A reversed phase HPLC using a Cyno column (Waters Sperisorb 250×4.6 mm, 10 μm) was developed for estimating the amount of salbutamol sulphate and ambroxol hydrochloride. The mobile phase was a variable mixture of buffer (1.0 ml of triethylamine in 1000 ml of water adjusted to pH 2.5 with orthophosphoric acid) and acetonitrile in the ratio 65:35 v/v. The mixture of equal volumes of water and acetonitrile was used as the diluent. The placebo and suitable dilutions of the standard and test solutions were injected into the column. The flow rate was 1.0 ml/min, detection wavelength was 276 nm, column temperature was ambient, injection volume was 100 μL and the run time was 15 min.
ii. Fine Particle Dose: The Fine particle dose was measured using the Apparatus D—Andersen Cascade Impactor of British Pharmacopoeia 2010 (A 330 Appendix XII C). The Apparatus was assembled with a pre-separator and a 1 μm glass fibre filter in place and the system was ensured to be airtight. The pre-separator contained 10 ml of diluent and each plate was coated with glycerine. The Apparatus was connected to a flow system and about 4 litres of air was drawn from the mouthpiece through the apparatus. The flow was adjusted to a value of 60±5 litres/min by means of a control valve. The pump was then switched off. The capsule was broken in the inhalation device (as described in FIG. 1) and with the pump running and the 2-way solenoid valve closed, the mouthpiece of the device was connected to the mouthpiece adapter of the Apparatus. The powder was discharged into the Apparatus by opening the 2-way solenoid valve for 5 seconds. The discharge sequence was repeated 4 more times. The Apparatus was dismantled and each stage was carefully washed with diluent and the washings were collected separately for each stage. The device and induction port washings were diluted to 50 ml, pre-separator washing was diluted to 100 ml, and the washings of the other stages was diluted to 25 ml with diluent.

The fraction of the total dose that was collected from Stage 2 to final filter was calculated as the FPD.

For calculating the amount of salbutamol sulphate in terms of salbutamol and ambroxol hyrochloride in terms of ambroxol in the washings, a reversed-phase HPLC method similar to the one mentioned for calculating DED was used.

### Examples 4-6

### Pharmaceutical Inhalation Compositions Containing Salmeterol Xinafoate and Fluticasone Propionate

| | Quantity (% w/w) | | |
|---|---|---|---|
| Ingredients | Example 4 | Example 5 | Example 6 |
| Salmeterol xinafoate | 0.30 | 0.30 | 0.30 |
| Lactose monohydrate | 7.72 | 7.72 | 7.72 |
| (Respitose SV010) | | | |
| (for loading salmeterol | | | |
| xinafoate) | | | |
| Water:Isopropanol (1:3 w/w) | q.s. | q.s. | q.s. |
| Fluticasone propionate | 0.40 | 1.00 | 2.00 |
| Lactose monohydrate | 3.60 | 9.00 | 18.00 |
| (Respitose SV010) | | | |
| (for loading for loading | | | |
| fluticasone propionate) | | | |
| Isopropanol:Methylene | q.s. | q.s. | q.s. |
| chloride (3:7 w/w) | | | |

Manufacturing Process:
1. Salmeterol xinafoate was dispersed in a sufficient quantity of a mixture of water and isopropanol.
2. Lactose monohydrate was loaded into a fluidized bed processor and the drug dispersions of Step I was coated onto the lactose monohydrate particles by top spray process to obtain granules.
3. Fluticasone propionate was dispersed in a sufficient quantity of a mixture of isopropanol and methylene chloride.
4. Lactose monohydrate was loaded into a fluidized bed processor and the drug dispersions of Step 3 was coated onto the lactose monohydrate particles by top spray process to obtain granules.
5. The granules of Step 4 were dried and sized through ASTM Sieve #200 to obtain particles.
6. The particles of Step 5 were filled in hard gelatin capsules so that each capsule contains 0.0725 mg of salmeterol xinafoate.

The composition of Example 4 was evaluated for deposition of emitted dose (DED) and fine particle dose (FPD) of salmeterol.

| Composition | Deposition of emitted dose | Fine particle dose |
|---|---|---|
| Example 4 | 29.05 | 33.95 |

Brief Analytical Procedure:
i. Deposition of Emitted Dose: The DED was calculated using the Apparatus A—Glass Impinger of the British Pharmacopoeia 2010 (A 324 Appendix XII C) The airflow was adjusted to 60±5 liters/min. The capsule containing the composition was broken in the inhalation device (as illustrated in FIG. 1) and was located in the mouthpiece of the Apparatus. The pump was switched on for 5 seconds. After switching off the pump, the device was removed. The discharge sequence was repeated 9 times. The inner surface of the inlet tube and the outer surface of the tube that projects into the lower impingement chamber were washed with the diluent and collected in the lower impingement chamber. The mixture of buffer (9.878 gm of ammonium acetate in 1000 ml of water, pH adjusted to 2.5±0.05 with ortho phosphoric acid), acetonitrile and methanol in the ratio 33:20:47 (v/v/v) was used as the diluent. The washings were diluted to 100 ml with the diluent and filtered through 0.45 μm filter and subjected to analysis.

A reversed phase HPLC using a Kromasil column (C-18, 150×4.6 mm, 5μ) was developed for estimating the amount of salmeterol xinafoate and fluticasone propionate. The mobile phase was a mixture of buffer, Acetonitrile and methanol in the ratio 33:20:47 (v/v/v). The mixture of equal volumes of water and acetonitrile was used as the diluent. The placebo and suitable dilutions of the standard and test solutions were injected into the column. The flow rate was 1.0 ml/min, detection wavelength was 278 nm, column temperature was ambient, injection volume was 50 μL and the run time was 15 min.
ii. Fine Particle Dose: The Fine particle dose was measured using the Apparatus D—Andersen Cascade Impactor of British Pharmacopoeia 2010 (A 330 Appendix XII C). The Apparatus was assembled with a pre-separator and a 1 μm glass fibre filter in place and the system was ensured to be airtight. The pre-separator contained 10 ml of diluent and each plate was coated with glycerine. The Apparatus was connected to a flow system and about 4 litres of air was drawn from the mouthpiece through the apparatus. The flow was adjusted to a value of 60±5 litres/min by means of a control valve. The pump was then switched off. The capsule was broken in the inhalation device (as illustrated in FIG. 1) and with the pump running and the 2-way solenoid valve closed, the mouthpiece of the device was connected to the mouthpiece adapter of the Apparatus. The powder was discharged into the Apparatus by opening the 2-way solenoid valve for 5 seconds. The discharge sequence was repeated 4 more times. The Apparatus was dismantled and each stage was carefully washed with diluent and the washings were collected separately for each stage. The device and induction port washings were diluted to 50 ml, pre-separator washing was diluted to 100 ml, and the washings of the other stages was diluted to 25 ml with diluent.

The fraction of the total dose that was collected from Stage 2 to final filter was calculated as the FPD.

For calculating the amount of salmeterol xinafoate in terms of salmeterol and fluticasone propionate in the washings, a reversed-phase HPLC method similar to the one mentioned for calculating the amount of drugs in DED was used.

### Example 7

### Pharmaceutical Inhalation Composition Comprising Salmeterol Xinafoate and Fluticasone Propionate

| | |
|---|---|
| Ingredients | Quantity (% w/w) |

Salmeterol xinafoate granules (A)

| | |
|---|---|
| Salmeterol xinafoate | 3.75 |
| Lactose monohydrate USP-NF | 96.25 |
| (Respitose SV010) | |
| Isopropanol | qs |
| Purified water | qs |
| Total for A | 100.00 |

Fluticasone propionate granules (B)

| | |
|---|---|
| Fluticasone propionate | 2.50 |
| Lactose monohydrate USP-NF | 97.50 |
| (Respitose SV010) | |
| Isopropanol | Qs |
| Methylene chloride | Qs |
| Total for B | 100.00 |

Final blend of powder composition

| | |
|---|---|
| Sized blend of (A) equivalent to 50 μg | 8.00 |
| salmeterol (based on assay) | |
| Sized blend of (B) equivalent to | 16.00 |
| 100/250/500 μg fluticasone (based on | |
| assay) | |

Manufacturing Process:
1. Salmeterol xinafoate was dispersed in a sufficient quantity of mixture of isopropanol and water.
2. Lactose monohydrate (sifted through ASTM sieve #60) was loaded into a fluidized bed processor and the drug solution of Step 1 was coated onto the lactose monohydrate particles by top spray process to obtain granules.
3. The granules were dried and sifted through ASTM sieve #200.
4. Fluticasone propionate was dispersed in a sufficient quantity of mixture of methylene chloride and isopropanol.
5. Lactose monohydrate (sifted through ASTM sieve #60) was loaded into a fluidized bed processor and the drug dispersions of Step 5 was coated onto the lactose monohydrate particles by top spray process to obtain granules.
6. The granules were dried and sifted through ASTM sieve #200.
7. The granules of step 3, granules of step 6 mixed together in a Turbula blender for 30 min at 30 rpm.
8. The blend thus formed was sifted through ASTM sieve #40.
9. The final blend formed of step 8 was filled in hard gelatine capsules.

The composition of Example 7 was evaluated for deposition of emitted dose (DED) and fine particle dose (FPD) of salmeterol and fluticasone using the analytical procedures and calculations described in Example 4 above.

Deposition of emitted dose for Example 7:

| | |
|---|---|
| Salmeterol + Fluticasone | Deposition of emitted dose (%) |

| (in μg) | Salmeterol | Fluticasone |
|---|---|---|
| 50 + 100 | 37.00 | 39.00 |
| 50 + 250 | 38.12 | 38.90 |
| 50 + 250 | 37.86 | 45.39 |

Fine particle dose for Example 7:

| | |
|---|---|
| Salmeterol + Fluticasone | Fine particle dose (%) |

| (in μg) | Salmeterol | Fluticasone |
|---|---|---|
| 50 + 100 | 27.89 | 29.25 |
| 50 + 250 | 28.22 | 29.55 |
| 50 + 250 | 27.88 | 29.54 |

### Comparative Examples A-C

### Compositions Containing Admixture of Salbutamol and Ambroxol

| | Quantity (% w/w) | | |
|---|---|---|---|
| Ingredients | Example A | Example B | Example C |
| Ambroxol | 8 | 16 | 24 |
| hydrochloride | | | |
| Salbutamol sulfate | 0.48 | 0.48 | 0.48 |
| Lactose monohydrate | 40.00 | 40.00 | 40.00 |
| (Respitose ML006) | | | |
| Lactose monohydrate | 51.52 | 43.52 | 35.52 |
| (Respitose SV010) | | | |
| Ethanol | q.s. | q.s. | q.s. |
| Purified water | q.s. | q.s. | q.s. |

Manufacturing Process:
1. Salbutamol sulphate, ambroxol hydrochloride and a first portion of lactose monohydrate (Respitose ML006) were sized through ASTM Sieve #60 and blended in geometric proportions in a blender for 15 min.
2. The blend of Step 1 was sized through ASTM Sieve #60 and mixed with a second portion of lactose monohydrate (Respitose ML006) and blended in a blender for 15 min.
3. The blend of Step 2 was sized through ASTM Sieve #60 and mixed with a first portion of lactose monohydrate (Respitose SV010) and blended in a blender for 15 min.
4. The blend of Step 3 was sized through ASTM Sieve #60 and mixed with a second portion of lactose monohydrate (Respitose SV010) and blended in a blender for 15 min.
5. The blend of Step 4 was sized through ASTM Sieve #60 and blended in a blender for 30 min.
6. The blend of Step 5 was filled in empty hard gelatin capsule so that each capsule contains 0.12 mg of salbutamol sulphate.

The compositions of Examples A-C were evaluated for deposition of emitted dose (DED) and fine particle dose (FPD) of salbutamol.

| | Deposition of | Fine particle dose |
|---|---|---|
| Composition | emitted dose (%) | (%) |
| Example A | 17.87 | 11.16 |
| Example B | 17.46 | 13.43 |
| Example C | 18.64 | 12.42 |

Brief Analytical Procedure:

The analytical procedures for calculating the DED and FPD were the same as that used for Examples 1-3.

We claim:

1.-21. (canceled)

22. A pharmaceutical powder composition for inhalation comprising an active ingredient and a pharmaceutically acceptable carrier, wherein the active ingredient is at least partially coated onto the carrier, and the composition has a deposition of emitted dose in the range of about 20% to about 75%.

23. The pharmaceutical composition according to claim 22, wherein the composition has a deposition of emitted dose in the range of 22% to 70%.

24. The pharmaceutical composition according to claim 22, wherein the composition has a deposition of emitted dose in the range of 25% to 60%.

25. The pharmaceutical composition according to claim 22, wherein the active ingredient includes a beta-2 adrenergic agonist, a steroid, an anti-cholinergic, a mucolytic or combinations thereof.

26. The pharmaceutical composition according to claim 22, wherein the active ingredient is selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof.

27. The pharmaceutical composition according to claim 22, wherein the pharmaceutically acceptable carrier is lactose.

28. The pharmaceutical composition according to claim 22, wherein the weight ratio of the active ingredient to the carrier ranges from about 1:2 to about 1:500.

29. A pharmaceutical powder composition for inhalation comprising an active ingredient and a pharmaceutically acceptable carrier, wherein the active ingredient is at least partially coated onto the carrier, and the composition has a fine particle dose in the range of about 20% to about 75%.

30. The pharmaceutical composition according to claim 29, wherein the composition has a fine particle dose in the range of 22% to 70%.

31. The pharmaceutical composition according to claim 29, wherein the composition has a fine particle dose in the range of 25% to 60%.

32. The pharmaceutical composition according to claim 29, wherein the active ingredient includes a beta-2 adrenergic agonist, a steroid, an anti-cholinergic, a mucolytic or combinations thereof.

33. The pharmaceutical composition according to claim 29, wherein the active ingredient is selected from a group consisting of salbutamol, salmeterol, formoterol, fenoterol, fluticasone, budesonide, mometasone, beclomethasone, ciclesonide, ambroxol, tiotropium, ipratropium, aclidinium or combinations thereof.

34. The pharmaceutical composition according to claim 29, wherein the pharmaceutically acceptable carrier is lactose.

35. The pharmaceutical composition according to claim 29, the weight ratio of the active ingredient to the carrier ranges from 1:2 to 1:500.

36. A process for preparing a pharmaceutical powder composition for inhalation, said process comprising: (a) dispersing an active ingredient in a solvent; (b) coating the dispersion of step (a) onto a pharmaceutically acceptable carrier to obtain a powder composition; (c) sizing the powder composition; and (d) optionally, blending the powder composition with the pharmaceutically acceptable carrier.
